(19) United States
(12) Patent Application Publication
(10) Pub. No.: US 2005/0120412 A1
(43) Pub. Date: Jun. 2, 2005

(54) Long day plants transformed with phytochrome characterized by altered flowering response to day length

(76) Inventor: Israela Wallerstein, Rehovot (IL)

Correspondence Address: G E Ehrlich;Anthony Castorina, Suite 207, 2001 Jefferson Davis Highway, Arlington, VA, 22202, US

(21) Appl. No.: 10/451,369

(22) PCT Filed: Dec. 24, 2001
(86) PCT No.: PCT/IL01/01192

Related U.S. Application Data
(60) Provisional application No. 60/259,170, filed on Jan. 3, 2001.

(51) Int. Cl. A01H 1/00; C12N 15/82

(52) U.S. Cl. 800/282

(57) ABSTRACT

A long day plant cultivated for commercial production of flowering-shoots, flowering pots, flowers, seeds or fruits is provided. The long day plant overexpresses a phytochrome protein such that the flowering-shoots, flowering pots, flowers, seeds or fruits thereof develop under substantially shorter natural days and lower sun irradiance than those required for development of the flowering-shoots, flowering pots, flowers, seeds or fruits in a similar long day plant not over expressing the phytochrome protein.

### FIELD AND BACKGROUND OF THE INVENTION

The present invention relates to long day plants characterized by altered flowering response to day length and to methods of generating same. More particularly, embodiments of the present invention relate to long day plants cultivated for commercial production of flowering-shoots, flowering pots, flowers, seeds or fruits which are characterized by altered responsiveness to day length, and to methods of producing such plants.

Many physiological processes in plants are known to be synchronized with the daily light cycle; some of these processes respond to the photoperiod length within this cycle, a phenomenon known as Photoperiodism.

Photoperiodism is characteristic of plants belonging to different taxonomic groups, such as monocotyledonous, dicotyledonous, perennials, annuals, bulb or corm forming plants.

Flowering response is one of many processes affected by photoperiodism. Depending on the plant species, flowering response can be affected in a qualitative (i.e. induction of flowering) or quantitative (i.e. acceleration of the flowering process) manner.

Plants in which the flowering process is induced or accelerated by day length can be divided according to a critical, maximal or minimal day length needed to induce or accelerate such flowering. For example, flowering of short day plants is induced or accelerated under a photoperiod shorter than a critical length whereas flowering of long day plants is induced or accelerated under a photoperiod longer than a critical length (Bemier et al, 1981).

There are plants in which distinct stages of the flowering process differ in the type of response to day length. In such plants a sequential application of varying day length (short followed by long or long followed by short) is needed to induce or accelerate their flowering process (Halevy A. H., 1985). Furthermore, there are long day plants which, in addition to a need for a critical day length, also respond to light intensity (light photon fluence rate) during one or more stages of their flowering process. In such plants, a higher light intensity will shorten the critical day length for the flowering response (Vince-Prue, 1994).

Plant photoreceptors which participate in the photoperiodism response to light, include the well characterized group of phytochromes. Phytochromes are a group of related proteins which are encoded by at least five divergent genes and which function as photoreceptors of light in a wavelength maximum of 660 ηm (Red=R) and 730 ηm (Far-Red=FR) of the photospectrum. The phytochromes respond to the ratio of R to FR in the light spectrum and to the photon fluence rate in this spectrum.

Phytochrome A (which is encoded by PHY A gene) and phytochrome B (which is encoded by PHY B gene) are members of this group. Phytochromes A and B are activated by Red light, while phytochrome A is also degraded under Red light and activated by Far-Red light. This difference in activation by light is associated with different modes of action for phytochromes A and B (Casal et al, 1996). In general, phytochromes may function as integral light-switchable components of transcriptional regulator complexes, permitting continuous and immediate sensing of changes in light signals directly at target gene promoters so as to allow control of the pattern of gene expression involved in determining photomorphogenic processes, including flowering (Murtas and Millar, 2000).

Phytochrome A or B expression levels affect various plant photomorphogenic responses, including flowering induction (Whitelam and Devlin, 1997). Phytochromes A and B participate in various processes, which enable the plant to sense a natural day cycle of light and dark. For example, phytochromes are responsible for measuring the length of a light period within a daily cycle and for responding to the transition between light and dark periods (Thomas and Vince-Prue, 1995: Somer et al, 1998: Samach and Coupland, 2000: Murats and Millar, 2000).

The role of phytochrome in regulating plant growth is of considerable commercial value in agriculture. As such, several prior art documents describe phytochrome-overexpressing plants, which present agronomic traits of commercial value.

For example, U.S. Pat. No. 5,268,526 to Hershey et al. describes the preparation of a transgenic plant overexpressing phytochrome of a monocotyledonous plant origin. The transgenic plant described by Hershey et al, exhibits a variety of useful agronomic traits such as reduced apical dominance, semidwarfism, increased shade tolerance, or dark green color.

U.S. Pat. No. 5,945,579 to Smith describes the generation of transgenic tobacco plants overexpressing phytochrome A, in which over expression confers an ability to undergo proximity-conditional dwarfing.

The role of phytochromes A and B in regulating the flowering response to day-length was studied in mutants lacking phytochrome expression (null mutants of either PHY A or PHY B) and in plants overexpressing phytochrome A or B. The role of phytochromes A and B in the regulation of the flowering response to day-length in long day plants was studied under artificial growth conditions which differed from natural conditions in light spectrum, light intensity and temperature. Under such artificial growth conditions a PHY A null mutant flowered later than wild type plants grown under long day conditions; although such PHY A deficiency did not change the flowering response to short-day conditions (Johanson et al, 1994: Weller et al, 1997: Reed et al, 1994: Whitelam and Harberd, 1994).

Flowering of Arabidopsis (a long day plant) overexpressing phytochrome A was studied also under tissue culture conditions which included artificial sterile agar medium, high humidity, constant temperature of 23° C., and fluorescent lighting of 200 μmol m⁻² s⁻¹ followed by day extension with 10 μmol m⁻² s⁻¹ from incandescent lamps. Under such conditions, overexpression of phytochrome A caused early flowering under long and short-day illumination conditions (Bagnall et al, 1995).

Under artificial growth conditions phytochrome B deficient Arabidopsis mutants (PHY B null mutants) flowered earlier than wild type plants, independent of day-length conditions (Putterill et al, 1995: Whitelam and Harberd, 1994: Goto et al, 1991: Reed et al, 1993), while overexpression of phytochrome B in Arabidopsis enhanced flowering only in tissue culture grown plants, both under long and short-day artificial lighting conditions (Bagnall et al, 1995).

Although various prior art studies regarding the effect of phytochrome overexpression on flowering describe long day plants in which early flowering was induced under artificial short or long day lighting conditions, such early flowering traits were induced under artificial lighting and growth conditions.

Artificial light conditions typically include white light provided from fluorescent lamps and/or far-red rich light provided from incandescent lamps. White light lacks the far-red spectrum while light from both incandescent and fluorescent lamps lacks the ultra violet spectrum. Furthermore, the intensity ratio between the different wavelengths comprising the artificial light used by these studies differs from that of sunlight.

In contrast, natural light (sunlight) is composed of various wavelengths of nearly equal intensities. In addition, sunlight varies in wavelength composition and intensity during dawn and dusk, cloudy or dusty days and according to the sun position at different seasons of the year.

In addition, under natural lighting conditions a temperature-light intensity relationship exists since high temperature is typically accompanied by high sun irradiance intensity and vice versa, whereas under artificial conditions, temperature and light intensity are not interdependent.

In most of the prior art studies described above, the artificial light intensities were much lower than sun light intensity, in the range of 60-200 μmol m⁻² s⁻¹ and were kept constant and irrespective of temperature throughout the experimentally induced short day conditions.

In addition, the growth conditions employed in the tissue culture experiments were extremely different from commercial growth conditions also in the root zone medium and the atmosphere composition within the tube.

Thus, although prior art long-day transgenic plants overexpressing phytochrome A are of some potential commercial value, at present long-day plants can only be made to flower earlier in the year under artificial lighting conditions and not under natural conditions desirable for commercial application of such early flowering trait.

There is thus a widely recognized need for, and it would be highly advantageous to have, phytochrome-overexpressing long-day plants cultivated for commercial production of flowering-shoots, flowering pots, flowers, seeds or fruits characterized by altered responsiveness to day length.

### SUMMARY OF THE INVENTION

According to one aspect of the present invention there is provided a long day plant cultivated for commercial production of flowering-shoots, flowering pots, flowers, seeds or fruits; the long day plant overexpressing a phytochrome protein in at least a portion of it's cells, such that the flowering-shoots, flowering pots, flowers, seeds or fruits thereof develop under substantially shorter days than that required for development of the flowering-shoots, flowering pots, flowers, seeds or fruits in a similar long day plant not overexpressing the phytochrome protein.

According to another aspect of the present invention there is provided a method of modulating a responsiveness of a long day plant to day length, the method comprising the step of overexpressing a phytochrome protein in at least a portion of the cells of the long day plant under conditions such that flowering-shoots, flowering pots, flowers, seeds or fruits of the long day plant develop under substantially shorter days than those required for development of the flowering-shoots, flowering pots, flowers, seeds or fruits in a similar long day plant not overexpressing the phytochrome protein.

According to further features in preferred embodiments of the invention described below, the long day plant overexpressing the phytochrome protein is derived from a commercial plant, plant derived tissue or a plant cell transformed with an exogenous expression cassette for overexpressing the phytochrome protein.

According to still further features in the described preferred embodiments the commercial plant, plant derived tissue or a plant cell is stably or transiently transformed with the expression cassette for overexpressing the phytochrome protein.

According to still further features in the described preferred embodiments the expression cassette forms a part of a nucleic acid construct selected from the group consisting of a DNA construct or an RNA construct.

According to still further features in the described preferred embodiments the exogenous expression cassette includes a phytochrome A or a phytochrome B encoding sequences.

According to still further features in the described preferred embodiments the exogenous expression cassette also includes a promoter sequence for directing expression of the phytochrome A or the phytochrome B encoding sequences in plant tissue.

According to still further features in the described preferred embodiments the promoter is selected from the group consisting of a constitutive promoter, an inducible promoter a developmentally regulated promoter and a tissue specific promoter.

According to still further features in the described preferred embodiments the long day plant overexpressing the phytochrome protein is a commercial dicotyledonous or monocotyledonous plant.

According to still further features in the described preferred embodiments the long day plant overexpressing the phytochrome protein is selected from the group consisting of an agronomic crop, an horticultural crop, and an ornamental plant.

According to still further features in the described preferred embodiments the long day plant overexpressing the phytochrome protein is an annual or a perennial plant selected from the group consisting of a rosette forming plant, a bulb forming plant, a corm forming plant, a herbaceous plant, a shrub forming plant and a tree forming plant.

According to still further features in the described preferred embodiments the flowering-shoots, flowering pots, flowers, seeds or fruits which develop under the substantially shorter days have at least one improved agronomic and/or commercial characteristic selected from the group consisting of an increased number of flowering shoots, an increased is number of flowers, an increased number of fruit forming flowers, a faster growth rate, a lower cold request for growth and flowering and reduced light intensity dependent flowering as compared to the similar long day plant not overexpressing the phytochrome protein.

According to still further features in the described preferred embodiments the day length of the substantially shorter days is at least 15% shorter than that required by the similar long day plant not overexpressing the phytochrome protein.

According to still further features in the described preferred embodiments the substantially shorter days are effected by natural lighting conditions.

According to still further features in the described preferred embodiments the substantially shorter days are further characterized by at least one condition selected from the group consisting of a light intensity between 80-2000 μmole m⁻²s⁻¹ PAR, and a temperature selected from the range between 5-30° C.

According to still further features in the described preferred embodiments the long day plant is cultivated for commercial production of flowering-shoots, flowering pots, flowers, seeds or fruits.

According to still further features in the described preferred embodiments the exogenous expression cassette for overexpressing the phytochrome protein is compatible for propagation in cells, or integration into the genome, of a plant.

According to still further features in the described preferred embodiments the step of overexpressing the phytochrome protein in the long day plant is effected by transforming the long day plant with an expression cassette encoding a phytochrome protein.

According to still further features in the described preferred embodiments the phytochrome protein is phytochrome A or phytochrome B.

According to still further features in the described preferred embodiments modulating the responsiveness of the long day plant to day length is utilized for producing flowering-shoots, flowering pots, flowers, seeds or fruits in the long day plant during substantially shorter days than those required by the long day plant for producing the flowering-shoots, flowering pots, flowers, seeds or fruits.

According to still further features in the described preferred embodiments modulating the responsiveness of the long day plant to day length is utilized for causing a spring or summer flowering plant to flower during autumn, winter or year-round.

According to still further features in the described preferred embodiments modulating the responsiveness of the long day plant to day length is utilized for conferring early flowering in the long day plant under short-day conditions.

The present invention successfully addresses the shortcomings of the presently known configurations by providing long day plants characterized by altered responsiveness to day length and methods of generating same.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates photoperiod and sun-irradiance effects on growth cycle length in Aster “Sun Karlo” plants transformed with PHY A cDNA. Greehouse grown plants were exposed to 10 hours daylight or to natural short day extended to 16 hours light by incandescent lighting (0.5 μmol m−2 s⁻¹ PAR. The time needed for inflorescence shoot elongation and flowering was measured during two growth cycles under different natural day conditions (Table 1). Grey and black columns indicate the first and second growth cycle, respectively.

FIG. 2 illustrates the time needed for inflorescence shoot elongation in Aster, “Sun Karlo” plants and transformed PHY A lines AA20 and AA21 and PHY B lines AB13-1 and AB12-6 under natural-day extension with incandescent lighting in the greenhouse. Plants were grown in the greenhouse under incandescent lighting (0.5 μmol m⁻² s⁻¹ PAR) at a day length extended to 14 or 16 h. Time needed for inflorescence shoot elongation to 55 cm in 90% of the plants was measured during two growth cycles under different natural day conditions (Table 1). Grey and black columns indicate the first and second growth cycle, respectively.

FIG. 3 illustrates the time needed for inflorescence shoot elongation in Aster, “Sun Karlo” plants and transformed PHY A lines AA20 and AA21 and PHY B lines AB13-1 and AB12-6 under the effect of natural day extension with fluorescent lighting. Plants were grown in the greenhouse under fluorescent lighting (0.5 μmol m⁻² s⁻¹ PAR) and extended day conditions of 14 or 16 h. Time needed for inflorescence shoot elongation to 55 cm in 90% of the plants was measured during two growth cycles under different natural day conditions (Table 1). Grey and black columns indicate the first and second growth cycle, respectively.

FIG. 4 illustrates the time needed for inflorescence shoot elongation in Aster, “Sun Karlo” plants and transformed PHY A lines AA20 and AA21 and PHY B lines AB13-1 and AB12-6, under natural-day conditions and night break with incandescent or fluorescent lighting. Plants were grown under greenhouse conditions and night-break treatments were applied at the middle of the night for two hours with either fluorescent or incandescent lighting (0.5 μmole m⁻² S⁻¹ PAR). Time needed for inflorescence shoot elongation to 55 cm in 90% of the plants was measured during two growth cycles under different natural day conditions (Table 1). Grey and black columns indicate the first and second growth cycle, respectively. FIG. 5 is photograph illustrating the effect of PHY A or PHY B overexpression on fruit development in transgenic Hypericum cv. “Excellent Flair” grown in a greenhouse under a gradual increase in natural day length (from February to June). At the end of this growth period, the transgenic lines (right side) exhibited enhanced flowering and fruit development, whereas the wild type plants (left side) were still at the beginning of the flowering stage and needed an additional growth period (one month) for fruit development.

FIGS. 6a-b are photographs illustrating the effect of PHY A or PHY B overexpression on inflorescent shoot development in transgenic Aster cv. “Sun Karlo” grown in a greenhouse under constant short days of 10 hours exposure to sun irradiance. At the end of this growth period, the transgenic lines overexpressing PHY A (FIGS. 6a-b left plants) produced inflorescent shoots, which is a typical response of Aster to long days, whereas the PHY B (FIGS. 6a-b middle plants) and the “Sun Karlo” control plants (FIGS. 6a-b right plants) produced rosette shoots, a typical response of Aster to short days.

FIG. 7 illustrates flowering and fruit setting in transgenic Hypericum plants overexpressing either PHY A or PHY B and grown under field conditions and natural day extension to 14 h of light by low intensity incandescent lighting. Non-flowering wild type “Excellent Flair” plants are on the left upper corner while flowering and fruit bearing transgenic plants are on the right lower corner of the photograph.

FIGS. 8a-g illustrate accumulated commercial yield of red-fruit bearing shoots harvested from various lines of Hypericum “Excellent Flair” plants overexpressing phytochrome under commercial field conditions. Commercial yields of transgenic plants (squares) overexpressing either PHY A (HA) or PHY B (HB) and the wild type cultivar “Excellent Flair” (EF, circles) were grown under natural day-length (solid line) or natural day-length extended to 14 h of light (broken line).

### DESCRIPTION OF THE PREFERRED EMBODIMENTS

The present invention is of methods of generating long day plants characterized by altered responsiveness to day length and of plants generated thereby which are of great commercial importance. In particular, the present invention can be utilized for generating commercial long day plants which can be cultivated for commercial production of flowering-shoots, flowering pots, flowers, seeds or fruits under short day conditions.

The principles and operation of the present invention may be better understood with reference to the accompanying descriptions.

Before explaining at least one embodiment of the invention in detail, it is to be understood that the invention is not limited in its application to the details of construction and the arrangement of the components set forth in the following description. The invention is capable of other embodiments or of being practiced or carried out in various ways. Also, it is to be understood that the phraseology and terminology employed herein is for the purpose of description and should not be regarded as limiting.

As used herein, the phrase “long day plant” refers to a plant, which initiates or accelerates the initiation of its flower formation following exposure to a day length longer than a critical day length. The critical day length is a specific feature of each plant but every plant which responds (in flowering) to days longer than its critical day length is considered a “long day plant”. Examples of long day plants include, but are not limited to, beet, radish, lettuce, spinach, Arabidopsis, Antirrhinum, Avena sativa, Pisum sativum, Hordeum vulgare, chrysanthemum, Brassica, Campanula, Delphinium, Dianthus, Fuchia, Gypsophilia, Helichrysum, Hyoscyamus, Jasminium, Lolium, Lunaria, Nicotiama sylvestris, Phlox, Salvia, Petunia, Trachelium, Trifolium, Triticum aestivum, Vicia faba and Sinapis alba (For additional examples, see Thomas and Vince-Prue, 1997).

As used herein, the phrase “shorter days” refer to days which include a lighting period which is substantially shorter than the critical day length required by a long day plant to flower. As is further described in the Examples section which follows, the lighting period of shorter days is 10-15% or more, shorter than that required for flowering in long day plants.

As used herein, the phrase “natural lighting conditions” refers to lighting which is identical to sunlight in its spectral components, intensity range, temperature to light-intensity relationship and/or seasonal dependence.

As used herein, the phrase “overexpressing a phytochrome protein” typically refers to generating phytochrome protein activity in some or all of the cells of a long day plant, which is substantially higher than the activity of this protein in a wild type plant grown under the same conditions. Typically, such over expression is effected by increasing the phytochrome concentration in the cell via stable or transient transformation with exogenous sequence(s), although upregulation of endogenous gene expression via “gene knock-in” of upregulatory sequences upstream to an endogenous phytochrome coding sequence is also envisaged.

The ability to control plant flowering and fruit development in commercially cultivated plants is of great importance to a grower. Such control can be exercised to increase flower and fruit yield, to grow crops under previously unsuitable conditions or to advance, delay or extend a growing season. As such, various prior art studies have attempted to generate plants characterized by early flowering. These studies have enjoyed limited success since such flowering depended upon conditions, which could not be reproduced in the field.

While reducing the present invention to practice, the present inventors have succeeded in generating long day plants which flower under short day conditions even when such conditions included commercial lighting conditions.

As used herein, the phrase “commercial lighting conditions” refers to growth conditions that include exposure to natural lighting conditions with or without addition of artificial lighting.

Thus according to one aspect of the present invention, there is provided a method of modulating a responsiveness of a long day plant to day length conditions, in particular modulating the responsiveness of long day plants to short day conditions which includes sun irradiance.

Preferably, shorter days are characterized by a lighting period shorter than a critical length, as well as specific spectral, light intensity and temperature conditions, since in a long day plant, light intensity and temperature conditions affect the critical day length, which becomes shorter with an increase in light intensity and/or temperature.

The method is effected by overexpressing a phytochrome protein in at least a portion of the cells of the long day plant under conditions such that flowering-shoots, flowering pots, flowers, seeds or fruits of the long day plant develop under substantially shorter days than those required for development, or for accelerated development, of the flowering-shoots, flowering pots, flowers, seeds or fruits in a similar long day plant not over expressing the phytochrome protein.

According to a preferred embodiment of the present invention, the step of overexpressing the phytochrome protein in the long day plant is effected by transforming at least a portion of the cells of the long day plant with an expression cassette including a phytochrome A or phytochrome B coding sequence being under the transcriptional control of a plant functional promoter. Any phytochrome A or phytochrome B encoding sequences can be utilized by the present invention including sequences derived from Oat, Cucurbita, Pea, Maize, Arabidopsis, Rice, Potato, Tobacco and any other non-angiosperm plant (see, for example Mathews and Sharrock, 1997 for additional details).

The plant functional promoter can be, for example, a constitutive promoter, such as for example, the Cauliflower Mosaic virus (CaMV) 35S promoter or the Ubiquitin promoter; an inducible promoter such as the tetracycline inducible promoter; or a developmentally regulated or tissue specific promoter.

Specific examples of suitable expression cassettes and expression construct harboring such cassettes are given in the Examples section, which follows.

Plant transformation using the phytochrome A or B expression cassettes described herein can be effected via any method known in the art for introducing nucleic acid constructs into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276). Such methods rely on either stable integration of the nucleic acid construct or a portion thereof into the genome of the plant, or on transient expression of the nucleic acid construct in which case these sequences are not inherited by a progeny of the plant.

There are two principle methods of effecting stable genomic integration of exogenous sequences such as those included within the nucleic acid constructs of the present invention into plant genomes:

(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.

(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

Following transformation plant propagation is exercised. Regeneration can be effected by seed propagation or vegetative propagation methods, which are well known in the art.

In addition to stable genomic expression, the phytochrome A or B expression cassettes can also be transiently expressed in a whole plant or in specific tissue regions thereof, including, for example, the shoot apical meristem (SAM) or leaves. Thus, in this case, transient transformation methods are utilized for transiently expressing the phytochrome A or B expression cassettes. Such methods include, but are not limited to, microinjection and bombardment as described above but under conditions which favor transient expression. For example, biolistic bombardment of shoot apical meristems can be utilized to transiently express phytochrome A or B therein.

In addition, packaged or unpackaged recombinant virus vector including the phytochrome A or B expression cassette can be utilized to infect plant tissues or cells such that a propagating recombinant virus established therein expresses phytochrome A or B either in a tissue restricted manner or in the entire plant (systemic infection).

Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al, Virology (1989) 172:285-292; Takamatsu et al. EMBO J. (1987) 6:307-311; French et al. Science (1986) 231:1294-1297; and Takamatsu et al. FEBS Letters (1990) 269:73-76.

According to another preferred embodiment of the present invention, the step of overexpressing the phytochrome protein in the long day plant is effected by “gene knock-in” of an exogenous polynucleotide encoding a transcriptional or translational enhancer.

Thus, for example, gene knock-in constructs including sequences homologous with regions upstream or downstream of the endogenous PHY A or B sequences can be generated and used to position transcriptional or translational enhancer sequence in cis regulatory control of the endogenous PHY A or B sequences to thereby upregulate expression of this gene.

These constructs preferably include positive and negative selection markers and may therefore be employed for selecting homologous recombination events. One ordinarily skilled in the art can readily design a knock-in construct including both positive and negative selection genes for efficiently selecting transformed plant cells that underwent a homologous recombination event with the construct. Such cells can then be grown into full plants. Standard methods known in the art can be used for implementing knock-in procedures. Such methods are set forth in, for example, U.S. Pat. Nos. 5,487,992, 5,464,764, 5,387,742, 5,360,735, 5,347,075, 5,298,422, 5,288,846, 5,221,778, 5,175,385, 5,175,384, 5,175,383, 4,736,866 as well as Burke and Olson, Methods in Enzymology, 194:251-270, 1991; Capecchi, Science 244:1288-1292, 1989; Davies et al., Nucleic Acids Research, 20 (11) 2693-2698, 1992; Dickinson et al., Human Molecular Genetics, 2(8):1299-1302, 1993; Duff and Lincoln, “Insertion of a pathogenic mutation into a yeast artificial chromosome containing the human APP gene and expression in ES cells”, Research Advances in Alzheimer's Disease and Related Disorders, 1995; Huxley et al., Genomics, 9:742-750 1991; Jakobovits et al., Nature, 362:255-261 1993; Lamb et al., Nature Genetics, 5: 22-29, 1993; Pearson and Choi, Proc. Natl. Acad. Sci. USA, 1993, 90:10578-82; Rothstein, Methods in Enzymology, 194:281-301, 1991; Schedl et al., Nature, 362: 258-261, 1993; Strauss et al., Science, 259:1904-1907, 1993, WO 94/23049, WO93/14200, WO 94/06908 and WO 94/28123 also provide information.

Thus, the method of the present invention can be utilized to modulate day length responsiveness in commercial dicotyledonous or monocotyledonous plants including, but not limited to, agronomic crop plants, horticultural crop plants, or ornamental plants. Examples of such plants include, but are not limited to, rosette forming plants such as Crysantheumum, Solidago, Solidaster, Gypsophyla, Trachelium Hyoscyamus, Lunaria and Scabiosa; bulb forming plants such as Allium, Lilium and Alstromeria; corm forming plants such as Aconitum, Anemone, Ranunculus, Liatris and Asclepias tuberosa; Herbaceous plants such as Anagallis, Campanula, Nigella, and Phlox; or Shrubs such as Fuchia, Hibiscus and Jasminium.

As is further described in the Examples section below, the plants generated according to the teachings of the present invention respond, in flowering, to short day conditions even when such conditions are provided by natural lighting utilized in commercial cultivation. Thus, unlike prior art methods, the method of the present invention is highly suitable for commercial applications since most commercial crops are grown in soil under such lighting conditions.

As is further described in the examples section which follows, the phytochrome overexpressing long day plant of the present invention is characterized by at least one improved agronomic and/or commercial characteristic including, but not limited to an increased number of flowering shoots, an increase number of flowers, an increased number of fruit forming flowers, a faster growth rate, a lower cold request for growth and flowering and a reduced light intensity dependent induction or acceleration of flowering as compared to the similar long day plant not overexpressing the phytochrome protein.

For example, a transgenic Hypericum cv. “Excellent Flair” plant generated according to the teachings of the present invention and grown under natural conditions over a growth period which covered winter through spring (in which natural day length is gradually elongated), reached the fruit development stage over a month before a similar non-transgenic plant (see the Examples section for further detail).

Transgenic Aster cv “Sun Karlo” plants generated according to the teaching of the present invention produced flowering shoots of commercial value under natural short days of winter in commercial greenhouse (see the Examples section below for further detail).

It will be appreciated that the above described characteristics of the transgenic long day plant of the present invention is of tremendous commercial value, since it enables the use of such a plant in commercial production of flowering-shoots, flowering pots, flowers, seeds or fruits under light conditions not suitable for such production from a wild type long day plant.

Thus, the method of the present invention would enable a grower to prolong a particular growing season of specific long day plants thereby generating higher yields from crops. In addition, growers at different parts of the world will be able to adopt new cultivars, which were previously restricted to specific geographical regions.

For example, plants which require a day length of over 13 hours for flowering will not flower under natural conditions in an equatorial zone unless their critical day length is shortened.

Further more, the method of the present invention will enable a grower to start cultivating a commercial crop earlier in the season or later toward the end of the season (depending on season), thereby enabling the grower to reach the marketplace earlier or to extend the market season of a particular plant product.

As is further described in the Examples section which follows, the response to day length varied between phytochrome A and phytochrome B overexpressing plants under the various lighting conditions tested. These variations enable the overexpression of a specific phytochrome for a specific purpose. For example, PHY A over expression can be utilized for year-round flowering, while PHY B overexpression can be utilized for extended flowering period and increased number of flowering shoots.

Thus, it will be appreciated that selective co-expression of both phytochromes in a long day plant can possibly be utilized to further enhance the plants response to various day length conditions.

For example, long day plants which overexpress both phytochrome A and B can be generated using double transformation techniques or by sexually crossing phytochrome A and phytochrome B expressing plants of the same cultivar. The phytochrome A and B encoding sequences can each be placed under the transcriptional control of a different induced promoter thereby enabling selective expression of one or both at any time during growth.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Generation of Agrobacterium Tumefaciens Harboring Phytochrome A or B Expression Cassettes

Phytochrome A Expression Cassette:

An Oat (Avena sativa) phytochrome A polynucleotide fragment which includes a “type 5” (GenBank Accession Number X03244) cDNA and “type 3” (GenBank Accession Number X03242) cDNA was provided in pFY122 (Boylan, M. T. and Quail, P. H., 1989, Plant Cell, 1: 765-773).

The oat phytochrome A polynucleotide fragment was excised from pFY122 and subcloned into the plasmid pROK2 generating the pRFYI plasmid vector (Smith Harry U.S. Pat. No. 5,945,579). The resultant pRFYI construct included the PHY A sequence subcloned downstream to the CaMV 35S promoter, and upstream to the polyadenylation signal sequences derived from the CaMV 35S transcript. The construct also included a bacterial selection marker for positive bacterial selection and a plant kanamycin-resistance coding sequence for positive plantlets selection (Beyan, M., 1984, Nucleic Acid Research, 12: 8711).

This pRFYI construct was used to transform Agrobacterium tumefaciens strain 2260 (Smith Harry U.S. Pat. No. 5,945,579) which was utilized for plant transformation in order to generate transgenic plants overexpressing phytochrome A.

Phytochrome B Expression Cassette:

An Arabidopsis phytochrome B (PHY B) coding sequence (GenBank Accession number X17342, Sharrock, R. A. and Quail, P. H. 1989. Genes Dev. 3, 1745-1757) was PCR amplified from a λEMBL3 clone (Somer, D. E., Sharrock, R. A., Tepperman, J. M. and Quail, P. H. 1991. The Plant Cell, 3, 1263-1274) and sequenced.

The PHY B PCR product was subcloned into pROK2 vector (Beyan, M. 1984. Nucl. Acids Res. 12, 8711-8721) to generate a pROKB plant expression vector in which PHY B is positioned in a sense orientation downstream to a CaMV 35S promoter and upstream to a polyadenylation signal. The pROKB construct further included a bacterial selection marker for positive bacterial selection and a plant kanamycin-resistance coding sequence for positive plantlets selection.

The pROKB construct was used to transform Agrobacterium tumefaciens strain LBA4404 (Halliday, K. J., Thomas, B. and Whitelam G. C., 1997; The plant Journal 12, 1079-1090) having a chromosomally located rifampicin resistance gene. The transformed Agrobacterium was utilized for plant transformation in order to generate transgenic plants overexpressing phytochrome B.

### Example 2

Generation and Analysis of Transgenic Aster cv. “Sun Karlo”

Aster plants were infected with Agrobacterium carrying expression cassettes for either phytochrome A or B in order to generate commercial long day plant characterized by an altered flowering pattern under short day conditions.

Plant Material:

Aster plants of cultivar “Sun Karlo” were grown in a controlled environment (phytotrone) in which sunlight was used as light source during at least a part of the photoperiod. In order to keep the plants in their rosette vegetative growth stage, the growing conditions were as follows:
(1) short-day lightning of 10 hours exposure to sun irradiance
(2) controlled temperatures of 20:12° C. during day:night periods respectively.

Plant Transformation:

Leaves of rosette shoots were surface sterilized in a 70% (v/v) ethanol for 1 minute followed by 10% (v/v) solution of domestic bleach for 8 minutes. Phytochrome carrying constructs were mobilized into the plant genome via Agrobacterium tumefacience infection. Leaf discs were excised and soaked in a 1/20 dilution of an overnight culture of the Agrobacterium strain containing the oat- PHY A-cDNA vector (pRFYI) or the Arabidopsis-PHY B-cDNA vector (PROKB) separately. Infected leaf discs were placed onto MS-salts medium plates containing 20 g/l sucrose, 0.1 mg/l naphthaleneacetic acid, 1.0 mg/l 6-benzylaminopurine and 7 g/l agar. Plates were incubated under constant temperature of 20° C. and 24-hour cycles of 16 hours low light intensity from cool white fluorescent lamps followed by eight hours of dark. Two days later, leaf discs were transferred onto fresh MS-salts medium plates containing 20 g/l sucrose, 0.1 mg/l naphthaleneacetic acid, 1.0 mg/l 6-benzylaminopurine and 7g/l agar with the addition of 100 mg/l kanamycin and 400 mg/l augmentin for selection.

Leaf discs were transferred onto similar fresh medium every two weeks until regenerated shoots were observed. The putative transgenic shoots were excised and transferred to MS-salts medium (MS salts supplemented with 20g/l sucrose, 100 mg/l kanamycin, 400 mg/l augmentin and 7g/1 agar). This medium was replaced every 2 weeks until roots developed.

The formation of roots by the excised shoots in the presence of kanamycin was an indication that the plants have been successfully transformed with the desired expression vector. Small plantlets (Shoots which developed roots) of approximately 1.0 cm to 2.0 cm in length were removed from the media and transplanted in soil containing pots for hardening in the phytotrone controlled environment. The selected controlled environment included: high humidity, short-day of 10 hours sun irradiance and controlled temperatures of 20:12 ° C., day:night, respectively.

Selection of Positive Transformed Aster Plants:

Leaves of hardened plants were PCR analyzed using forward primer 5′-CGCCTTCTGGCTATCAGATG -3′ (SEQ ID NO: 1) and reverse primer 5′-CGAGGAAGCATTGCTACTGT-3′ (SEQ ID NO: 2) specific to the Oat PHY A sequence, and forward primer 5′-GCTTGTTCCAGCAAGGACTACT-3′ (SEQ ID NO: 3) and reverse primer 5′-GCTGCTATGGAACATGTCT-3′ (SEQ ID NO: 4) specific to the Arabidopsis PHY B coding sequence.

PCR positive plants were further analyzed for expression of the exogenous cDNAs. This was performed by employing reverse transcription-PCR (RT-PCR) analysis on mRNA derived from the transformed plants. For the reverse-transcription step, a 12-18 mer oligo (dT) primer (GibcoBRL Cat #. 18418-012) was used in the presence of the reverse-transcriptase superscript II (Gibco-BRL Cat #18064-022). Primers specific to phytochrome cDNA (SEQ ID NOs:1 and 2 or 3 and 4) were used in the PCR amplification step following the RT step.

Positive selected plants were vegetatively propagated. Cuttings of rosette shoots were rooted under phytotrone controlled environment which included short-days of 10 hour sun irradiance and temperatures of 20:12° C. day:night, respectively.

Three generations of plants of each transformant were grown prior to conducting physiological experiments designed for assessing the response of the Aster transgenic plants to day-length.

Cultivation Conditions:

Day-length treatments under phytotrone or greenhouse conditions were applied to transgenic Aster (cv “Sun Karlo”) carrying PHY A or PHY B coding sequences.

Phytotrone experiment: phytotrone conditions were as follows: a controlled temperature of 20:12° C., day:night respectively; lightning conditions of 8 hour exposure to sun irradiance (400 μmolm⁻² s⁻¹ PAR) followed by day extension treatments of 2 hours which were applied either with sun irradiance or with artificial light from incandescent plus fluorescent lamps. The artificial light intensities were as follows: 20 μmol m⁻²s⁻¹ PAR, 8.1 μmol m⁻²s⁻¹ in the red (600-700 ηm) and 8.0^{˜} μmol m⁻²s⁻¹ in the far-red (700-800 ηm) spectra with R:FR ratio of 1.02, similar to that of sun irradiance. Night break treatments included short lighting, from incandescent or fluorescent lamps, for 15 or 30 minutes which were introduced in the middle of the 16 hours dark period. The incandescent lamp light intensity was 1.4 μmol m⁻²s⁻¹ PAR, 0.9 μmol m⁻² s⁻¹ red (600-700 ηm) and 1.5 μmol m⁻²s⁻¹ far-red (700-800 ηm) with R:FR ratio of 0.6. Fluorescent lamp light intensity was 2.4 μmole m⁻²s⁻¹ PAR, 1.1 μmole m⁻² s⁻red (600-700 μm) and 0.1 5 μmole m⁻²s⁻¹ far-red (700-800 ηm), with R:FR ratio of 7.3.

Greenhouse experiment: the greenhouse experiment started at September, and included two growth cycles, which ended in April.

Growth conditions were as follows: a temperature of 25:14° C., day: night, respectively. Natural short days and sunlight conditions were used as the basis for day extension and night break treatments with artificial lighting. Several different treatment were applied as follows:
(i) a 10 hours exposure to sun irradiance was applied constantly during the entire growth cycle; or
(ii) each growth cycle started with long-day conditions during inflorescent-shoot elongation followed by natural day conditions during flower development.

Long-day conditions included day extension or night break treatments, which were applied with incandescent or fluorescent lamps having a light intensity of 0.5 μmole m⁻²s⁻¹ PAR. Natural day lightning was extended to light-period (photoperiod) of 14 or 16 hours. Alternatively, natural day lightning was followed by a night break of 2 hours. The long day treatments were applied during inflorescent shoot elongation until a shoot length of 50-55 cm was reached. When 90% of the shoots reached this length the plants were transferred to natural day length conditions for flower initiation and flowering. At flowering, shoots were cut back to soil surface and the plants were transferred back to long day conditions for the second growth cycle which started from rosette shoots.

### Example 3

### Response of the Transgenic Aster to Various Day-Length Treatments

Critical Day Length in Transgenic Aster Plants:

Table 1 below represents the effect of day extension treatments in the phytotrone on the transition from rosette to inflorescent shoot development and flowering. As mentioned above the plants were exposed to sun irradiance for eight hours. Two hours of day extension were applied either by additional sun irradiance (high light intensity) or artificial light from incandescent and fluorescent lamps (low light intensity).

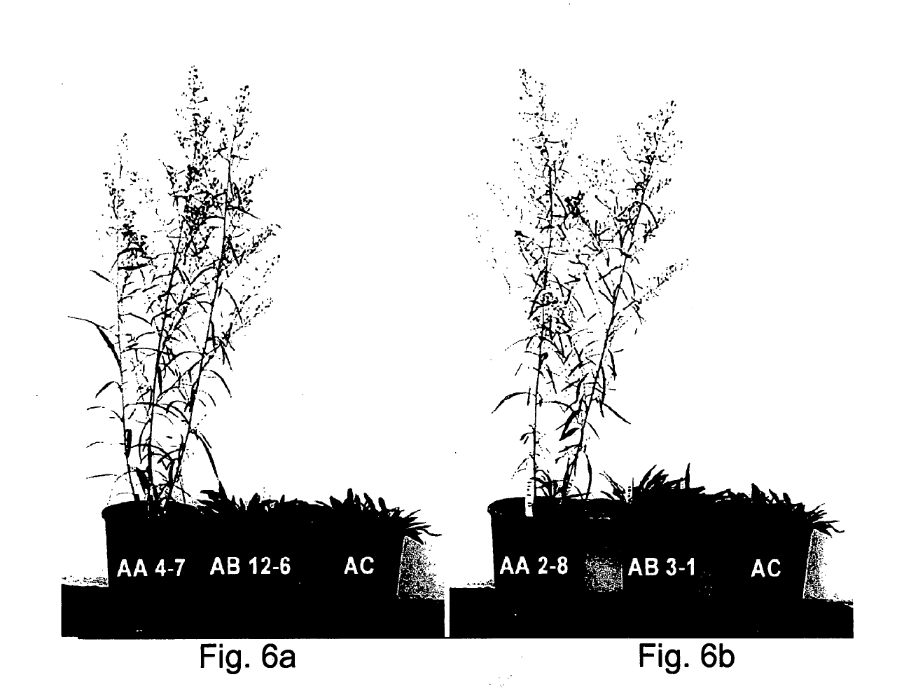

According to the data in Table 1 and as shown in FIG. 6, overexpression of phytochrome A considerably shortened the critical day length and enabled inflorescent shoot development and flowering in the transgenic line AA4-7 to occur under day length of eight hours sun irradiance. Day length of 10 hours was short for non-transgenic plants (“Sun Karlo”) and therefore their inflorescent shoots did elongate and as a consequence did not flower. Similarly, day length of 10 hours was short for plants overexpressing phytochrome B (data not shown).

Another PHY A overexpressing transgenic line: AA17-3 had critical day length of 10 hours. Its inflorescent shoots did not develop under day length of eight hours, but when the day length was extended from eight hours to ten hours, transgenic line AA17-3 developed inflorescent shoots. The difference in critical day length between the two PHY A overexpressing lines and between the transgenic and non-transgenic lines indicates that overexpression of phytochrome A does not eliminate the requirement for long days but shortens the critical day length needed for a long day effect. As a consequence, days shorter than an original critical day length are perceived as long days by the transgenic plants of the present invention.

The number of rosette leaves produced prior to shoot elongation indicated the physiological time of transition from rosette to inflorescent-shoot elongation (the beginning of the flowering process), a typical response of Aster to long day conditions. The number of rosette leaves produced on transgenic and non-transgenic plants under the day extension treatments further indicated that PHY A overexpression did not eliminate the effect of day length and light intensity on the transition process but enabled its operation under a substantially shorter day length and lower light intensity. While “Sun Karlo, the non-transgenic line, continuously produced rosette leaves under eight or ten hours of light independent of light intensity during day extension, the PHY A overexpressing lines: AA4-7 and AA17-3 accelerated the transition process (produced less rosette leaves prior to the transition) as a response to an increase in day length and/or light intensity.

Between the two PHY A lines AA4-7 was less sensitive to the photoperiod treatments and to the difference between day extension with artificial lighting and that with sun irradiance indicating lower sensitivity of the PHY A overexpressing plants to light intensities.

TABLE 1
Day-length and sun-irradiance effects on inflorescent
shoot and flower development in Aster “Sun Karlo” plants transformed
with PHY A cDNA. Plants were exposed to eight hours sun irradiance
extended to ten hours by sun or artificial lighting.

| | | Time to | Rosette leaves at |
|---|---|---|---|
| Transgenic | Light source for day | flowering | flowering |
| line | extension | Days | No. |
| AA 4-7 | Non | 72 ± 0.8 | 6.8 ± 0.5 |
| AA 4-7 | Lamps | 76 ± 2.0 | 5.2 ± 0.4 |
| AA 4-7 | Sun | 77 ± 0.4 | 4.8 ± 0.4 |
| AA 17-3 | Non | | 12.2 ± 1.0 |
| AA 17-3 | Lamps | 88 ± 3.7 | 6.5 ± 0.7 |
| AA 17-3 | Sun | 95 ± 4.5 | 3.8 ± 0.5 |
| Sun Karlo | Non | | 15 ± 1.2 |
| Sun Karlo | Lamps | | 14.8 ± 1.8 |
| Sun Karlo | Sun | | 15.7 ± 0.9 |

Night Break Effect on Transgenic and “Sun Karlo” Aster Plants

Incandescent Lighting:

Light applied in the middle of the night (night break) has a long day effect on flowering in long day plants. To affect flowering in long day plants the length of the night break should exceed a minimum length. Table 2 below represent the effect of an eight hour exposure to sun irradiance (short day) followed by 15 or 30 minutes of night break with incandescent lighting on inflorescent shoot development in transgenic plants overexpressing phytochrome A or B. Light from incandescent lamp includes both red and far-red spectrum but is relatively rich in the far-red spectrum.

Night break treatments (15 or 30 minutes) were too short for the non-transgenic “Sun Karlo” plants and therefore they did not develop inflorescent shoots but continued to produce rosette leaves. This night break duration, induced inflorescent shoot development and flowering in transgenic lines overexpressing either PHY B: AB12-6 and AB3-1 or PHY A: AA17-3. The PHY A line AA4-7, which developed inflorescent shoot under eight hours exposure to sun irradiance (see Table 1), accelerated the transition from rosette to inflorescent shoot (produced less rosette leaves) in response to this night break treatment.

The effect of night break on the PHY B lines: AB12-6 and AB 3-1 was higher than the effect of day extension and therefore plants that did not develop inflorescent shoots under day length of 10 hours (Table 1) did so under 15 minutes night break (Table 2). Overexpression of either phytochrome A or B did not eliminate the plants response to the night break duration as indicated by the lower number of rosette leaves they produced under the longer night break. However, these results clearly demonstrate that phytochrome A or B overexpressing plants respond to limited long day treatments (short night breaks), which have no effect on non-transformed plants.

TABLE 2
Inflorescent-shoot and flower development on Aster “Sun Karlo”
plants transformed with Oat-PHY A or Arabidopsis-PHY B. Plants were
treated with eight hours exposure to sun irradiance followed by short
night break with incandescent lighting in the phytotrone.

| | | | Rosette |
|---|---|---|---|
| | | Time to | leaves at |
| Transgenic | Night break duration | flowering | flowering |
| lines | Min. | Days | No. |
| AA 4-7 | 15 | 76 ± 1.0 | 5.0 ± 0.4 |
| AA 4-.7 | 30 | 77 ± 1.5 | 3.8 ± 0.2 |
| AA 17-3 | 15 | — | 10.7 ± 1.1 |
| AA 17-3 | 30 | 98 ± 2.0 | 5.5 ± 0.6 |
| AB 12-6 | 15 | 99 ± 4.6 | 8.6 ± 0.8 |
| AB 12-6 | 30 | 99 ± 2.6 | 5.5 ± 0.6 |
| AB 3-1 | 15 | 101 ± 1.0 | 4.8 ± 0.8 |
| AB 3-1 | 30 | 100 ± 2.6 | 4.8 ± 0.2 |
| “Sun karlo” | 15 | — | 12.3 ± 0.9 |
| “Sun karlo” | 30 | — | 13.0 ± 2.1 |

Fluorescent Lighting:

The night break lighting conditions described above was also provided from fluorescent lamps, which produce the red but not the far-red spectrum.

As is shown in Table 3, elimination of far-red light did not change the response of “Sun Karlo' plants to the short night break and therefore they continued to produce rosette leaves and did not flower. The PHY B overexpressing lines: AB12-6 and AB3-1 responded to the short night break treatments with fluorescent lighting in inflorescent shoot development and flowering and to the increased duration of lighting with reduced number of rosette leaves. The PHY A overexpressing line: AA17-3, which responded to 30 minutes of night break with incandescent light in inflorescent shoot development and flowering, did not respond to similar night break duration when applied via fluorescent lighting. This specific response to far-red rich light is typical of active phytochrome A indicating stable expression of PHY A cDNA in the transgenic plants of the present invention.

A similar conclusion can by drawn for the PHY B transgenic lines, which responded to night break treatments provided by both incandescent or fluorescent lamps.

TABLE 3
Inflorescent-shoot and flowers development on Aster “Sun
Karlo” plants transformed with oat-PHY A or Arabidopsis-PHY B. Plants
were treated with eight hours exposure to sun irradiance followed by short
night break with fluorescent lighting in the phytotrone.

| | Night break | | Rosette |
|---|---|---|---|
| Transgenic lines | duration | Time to flowering | leaves at flowering |
| ID | Min. | Days | No. |
| AA 4-7 | 15 | 80 ± 1.3 | 6.4 ± 0.6 |
| AA 4-.7 | 30 | 67 ± 1.6 | 4.7 ± 0.2 |
| AA 17-3 | 15 | — | 12.8 ± 0.4 |
| AA 17-3 | 30 | — | 13.9 ± 0.4 |
| AB 12-6 | 15 | 82 ± 2.6 | 6.5 ± 0.5 |
| AB 12-6 | 30 | 64 ± 1.6 | 4.9 ± 0.5 |
| AB 3-1 | 15 | 77 ± 1.7 | 7.4 ± 0.8 |
| AB 3-1 | 30 | 57 ± 2.1 | 6.0 ± 0.4 |
| “Sun karlo” | 15 | — | 15.5 ± 0.7 |
| “Sun karlo” | 30 | — | 16.8 ± 1.2 |

### Example 4

### Development of Inflorescent Shoots in Transgenic Aster Plants Under the Influence of Natural Sun Irradiance Conditions in the Greenhouse

As mentioned above, the intensity of sunlight affects the critical day length in some long day plants including Aster. To test the effect of sun irradiance on the rate of inflorescent shoot development, phytochrome overexpressing “Sun Karlo” plants were grown for two cycles in a greenhouse under a constant photoperiod but with varying sun irradiance conditions. The natural day length was shorten to constant photoperiod of 10 hours or extended to 14 or 16 hours. The natural sun irradiance conditions during inflorescent shoot development in two growth cycles are presented in Table 4 below. As can be seen therein, in the first growth cycle, the natural day length was longer, and the sun irradiance was higher than in the second growth cycle.

TABLE 4
Natural day length and sun irradiance conditions during the period
of inflorescent shoot development in two growth cycles of transgenic
and non-transgenic Aster cv. “sun Karlo” in the greenhouse.

| | Day-length | Sun-irradiance |
|---|---|---|
| | h:min | μmole m⁻²s⁻¹ |
| First growth cycle | 12:00-10:40 | 1000-500 |
| Second growth cycle | 10:06-11:00 | 300-500 |

Natural Day Extension with Incandescent Lighting:

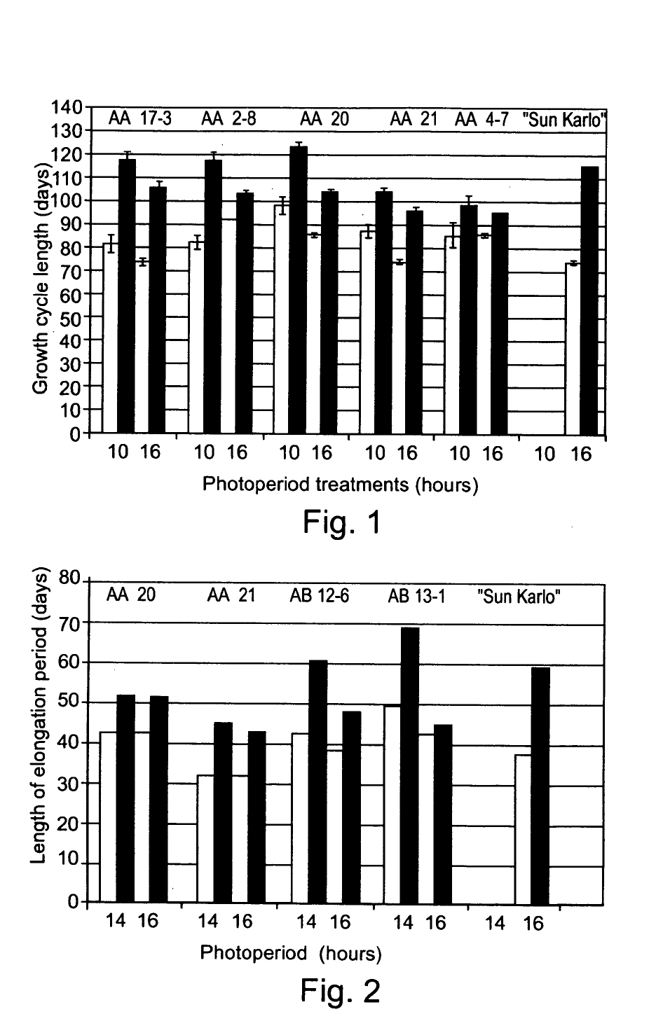

As shown in FIG. 1, the non-transgenic “Sun Karlo” plants were highly responsive to photoperiod and sun irradiance conditions provided in the greenhouse. These plants did not develop inflorescent shoots under photoperiod of 10 hours, independent of sun irradiance intensity. However under photoperiod of 16 hours their first growth cycle was considerably shorter than the second one.

In the greenhouse (FIG. 1) as well as under the phytotrone conditions overexpression of PHY A shortened the critical day length, therefore the PHY A overexpressing lines: AA17-3, AA2-8, AA20, AA21 and AA4-7 developed inflorescent shoots and flowers during the two growth cycles under constant day length of 10 hours.

Under the 10 hour photoperiod the effect of sunlight intensity on the growth cycle length (the difference between the length of the first and the second growth cycle) differed among the PHY A overexpressing lines: AA17-3, AA2-8, AA20, AA21 and AA4-7. Overexpression of phytochrome A almost overcame the effect of sunlight intensity on shoot development in the line AA4-7 and its growth cycles were nearly unaffected by sun irradiance conditions as well as photoperiod.

Under a photoperiod of 16 hours the difference between the length of the two growth cycles was higher in “Sun Karlo” than in the four PHY A transgenic lines AA2-8, AA20, AA21 and AA4-7. These PHY A overexpressing lines will produce flowering shoots not only under substantial short days but also under low sun irradiance, thus enabling flowering under winter conditions.

FIG. 2 describes the time needed for transgenic and “Sun Karlo” plants to develop 50 cm length of inflorescent shoots under photoperiods of 14 and 16 hours, based on natural day extension with incandescent lighting.

“Sun Karlo” plants developed inflorescent shoots only under natural day extension to 16 hours, with considerable effect of sun irradiance conditions on the rate of this development (the difference in length between the two growth periods).

As demonstrated above the critical day length of PHY A overexpressing lines was shorter than 10 hours and thus it is not surprising that they developed inflorescent shoots under a photoperiod of 14 or 16 hours with no photoperiod effect on the developmental rate. Little sun irradiance effect on the developmental rate, during the two growth cycles and under the two photoperiods, was observed.

The PHY B overexpressing lines: AB12-6 and AB13-1 had critical day length longer than 10 hours but shorter than 14 hours and therefore they did not develop inflorescent shoots under a photoperiod of 10 hours, but did so under a photoperiod of 14 hours. The effect of sun irradiance on the rate of their inflorescent shoot development was higher under a photoperiod of 14 than 16 hours. Under natural day extension to 16 hours the rates of inflorescent shoot development in transgenic PHY B lines were only slightly affected by the natural day sun irradiance conditions, meaning that low sun irradiance during winter will have a slight effect on flowering in these lines under day extension conditions.

Natural Day Extension with Fluorescent Lighting:

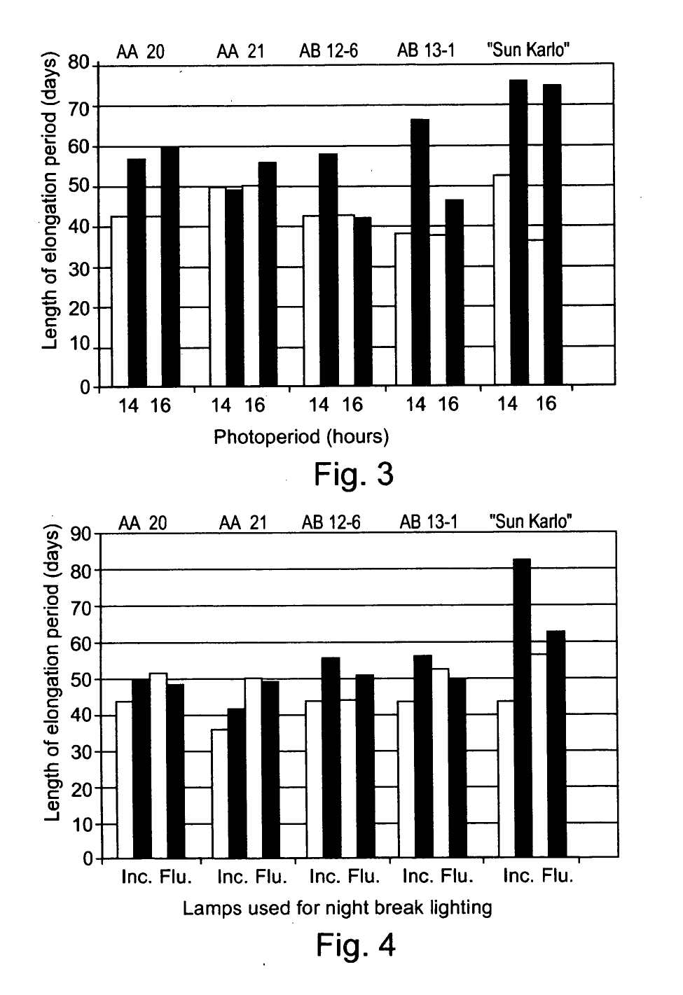

FIG. 3 demonstrates that in non-transgenic “Sun Karlo” plants phytochrome plays an important role in determining the critical day length. “Sun Karlo” plants responded not only to a photoperiod length and sun irradiance conditions, but also to the light spectrum during day extension when the photoperiod was extended to the critical day length of these plants. When natural day was extended to 14 hours with incandescent lighting, the “Sun Karlo” plants did not develop inflorescent shoots whereas extension with fluorescent lighting induced the development of inflorescent shoots. The developmental rate of these shoots was highly influenced by sun irradiance conditions during the two growth cycles.

The PHY A overexpressing lines that were relatively insensitive to day length longer than 10 hours showed little difference in their response to day extension with incandescent or fluorescent lighting.

The PHY B overexpressing lines responded similarly to day extension or night break treatments provided by fluorescent or incandescent lighting.

Night Break Treatment:

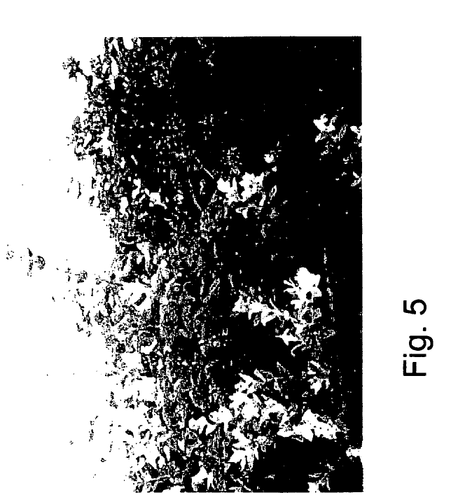

FIG. 4 demonstrates that two hours of night break with either incandescent or fluorescent lighting induced inflorescent shoot development in “Sun Karlo” plants. The developmental rate of these plants when treated with incandescent lighting was highly influenced by sun irradiance conditions whereas fluorescent lighting treatment overcame the sun irradiance effect (almost no difference between the developmental rate during the two growth cycles).

The transgenic PHY A or B overexpressing plants, which responded to the short night break (15 minutes in the phytotrone, see above) where almost insensitive to spectral content when night break was applied for two hours. Thus, the sun-irradiance effect on the developmental rate of inflorescent shoots even under night break with incandescent lighting was eliminated from PHY A overexpressing lines: AA20 and AA21 and PHY B overexpressing lines: AB12-6 and AB13-1.

### Example 5

### Comparison of Commercial Parameters Regarding the Yield of Aster Transgenic Plants Grown in a Commercial Greenhouse During Wintertime in Israel

PHY A or B transgenic lines and “Sun Karlo” non-transformed Aster plants were grown in a commercial greenhouse for cut-flower production during the winter in Israel.

Growth conditions were as follows:

Natural short-day conditions were applied to the PHY A transgenic plant lines AA2-8 and AA4-7 while, natural short-day conditions extended by incandescent lighting to a photoperiod of 14 or 16 hours were applied during inflorescent shoot elongation to a “Sun Karlo” non-transformed Aster plant, the PHY A transgenic line AA2-8 and the PHY B transgenic lines AB3-1 and AB12-6. In all cases, the inflorescent-shoot served as the “cut-flower”. Cuttings of flowering shoots were performed at the end of each growth cycle. Three successive growth cycles were performed between autumn and spring in Israel.

The commercial quality of the flowering shoots, measured by their length and weight, was good and almost the same in all treatments; the number of flowering shoots was highly affected by plant transformation. Overexpression of either, PHY A or B increased the number of flowering shoots mainly in the second and third growth cycles (Table 5 below). PHYA overexpression in the AA2-8 line increased the total shoot yield by 51% without day extension and by 115% with minimum day extension. PHY B expressing lines AB3-1 and AB12-6 exhibited an increase in the total shoot yield of 94% under day extension to 14 and 16 h, respectively. This commercial-yield test proves that transformation of Aster with Phytochrome encoding cDNA can increase the yield of flowering shoots and eliminate or reduce the need for artificial lighting when natural days are short and during year-round production of “cut-flowers”.

TABLE 5
Flowering shoots (“cut flowers”) yield in wild type Aster cultivar
“Sun Karlo” and four transgenic lines grown under commercial conditions
from autumn through spring

| | | Flowering | | Flowering | | |
|---|---|---|---|---|---|---|
| | Photo- | shoots | Flowering shoots | shoots | Total shoot | |
| | period | First cycle | second cycle | third cycle | yield | Total |
| Transgenic line | hours | No./m² | No./m² | No./m² | No./m² | shoots yield % |
| Wild type | 16 | 26 | 32 | 114 | 172 | 100 |
| AA2-8 | Natural | 33 | 64 | 164 | 261 | 151 |
| AA2-8 | 14 | 28 | 92 | 251 | 371 | 215 |
| AA4-7 | Natural | 33 | 41 | 141 | 215 | 125 |
| AB3-1 | 14 | 39 | 75 | 222 | 336 | 195 |
| AB12-6 | 16 | 29 | 75 | 231 | 335 | 195 |

Natural daylength changed during the production period between 13 h:40 min and 10 h. Day extension was effected by incandescent lighting. During this period three growth cycles and flowering shoots cutting were performed. The four transgenic lines included the PHY A overexpressing lines AA2-8 and AA4-7, and the PHY B overexpressing lines AB3-1 and AB12-6.

### Example 6

### Generation and Analysis of Transgenic Hypericum cv. “Excellent Flair” Plants

Plant Material:

Hypericum cv. “Excellent Flair” plants were grown in the phytotrone controlled environment in order to keep the plants in their vegetative growth state. Growth conditions included short-day conditions of 10 hours exposure to sun irradiance and controlled temperatures of 17:9° C. during day:night, respectively.

Plant Transformation:

Fully expanded leaves were surface sterilized in a 70% (v/v) ethanol for one minute followed by 10% (v/v) solution of domestic bleach for eight minutes. Cut leaf petioles were soaked in a 1/20 dilution of an overnight culture of the desired Agrobacterium strain containing either the oat-PHY A-cDNA expression vector (pRFYI) or the Arabidopsis-PHY B-cDNA expression vector (pROKB) described hereinabove.

Infected petioles were placed onto MS salts plates, which contained half concentration of MS salts (0.5×MS) supplemented with 20g/l sucrose, 0.5 mg/l naphthaleneacetic acid, 2.0 mg/l 6-benzylaminopurine and 7g/l agar. Plates were incubated under a constant temperature of 20° C. and 24 hours cycles of 16 hours low light intensity from cool white fluorescent lamps followed by eight hours of dark.

Two days following transformation, the petioles were transferred to fresh MS salts plates containing 0.5×MS salts, 20g/l sucrose, 0.5 mg/l naphthaleneacetic acid, 2.0 mg/l 6-benzylaminopurine, 7g/l agar, 100 mg/l kanamycin and 400 mg/l augmentin and further incubated therein for three weeks.

For shoot regeneration, the petioles were transferred onto fresh medium containing 0.5×MS salt concentration, 20 g/l sucrose, 7 g/l agar, 0.5 mg/l gibberellic acid, 1.0 mg/l 6-benzylaminopurine, 0.05 mg/l indolebutiric acid, 100 mg/l kanamycin and 400 mg/l augmentin. Regenerated shoots were excised and transferred onto fresh 0.5×MS plates supplemented with 20 g/l sucrose, 7 g/l agar, 100 mg/l kanamycin and 400 mg/l augmentin).

The formation of roots in the excised shoots in the presence of kanamycin was an indication that the plants have been successfully transformed with the desired expression vector. Small plantlets (Shoots which developed roots) of approximately 1.0 to 2.0 cm in length were removed from the media and transplanted in soil containing pots for hardening under phytotrone controlled conditions, which included high humidity, short-days of 10 hours sun irradiance and controlled temperatures of 20° C. day and 12° C. night.

Selection of Positive Transformed Hypericum Plants:

Leaves of hardened plants were PCR analyzed and the PCR positive plants were further analyzed for expression of the exogenous cDNAs as described hereinabove.

Positive selected plants were vegetatively propagated, using young shoots as cuttings. These plants were grown under phytotrone controlled environment which included short-day conditions for rooting. Before testing the response of the Hypericum transgenic plants to day-length, three generations of plants originating from rooting shoots were grown. Each successfully grown plant generation represent a stable transformed plant expressing the relevant phytochrome coding sequences.

Cultivation Conditions:

Greenhouse/phytotrone: PHY A or PHY B transgenic plants of Hypericum cv. “Excellent Flair” were exposed to controlled environmental conditions either in the phytotrone or the greenhouse. Phytotrone conditions included temperature of 23:15° C., day:night, respectively, and short natural day illumination conditions of 10 hour exposure to sun irradiance (800 μmol m⁻²s⁻¹ PAR). These conditions prevented the non-transgenic Hypericum plants from flowering and setting fruits. In the greenhouse a gradual increase in natural day-length starting in February (10 h:40 min) and ending in June (14 hours), was utilized to select early flowering plants.

Field: The transgenic Hypericum plants described above were also grown in the field under commercial growth conditions. Seedlings were planted in the field on October 1 and cut-back to fifth internode on October 25. From October 25 to June 16, the plants were grown under lighting conditions which included a natural change in day-length as follows: 11 h:06 min on October 25 decreasing to 10 h:03 min on December 21and increasing to 14 h in June; or a natural day extension of up to 14 h via incandescent lighting (0.7 μmol m⁻² s⁻¹ PAR).

### Example 7

### Flowering and Fruit Development in the Transgenic Hypericum Plants

Data pertaining to flower development in the transgenic Hypericum, cv “Excellent Flair” plant lines is summarized in Table 6 below.

TABLE 6
Flowering of transgenic Hypericum, cv “Excellent Flair”
transformed with oat-PHY A or Arabidopsis-PHY B coding sequences
under short days in the phytotrone or greenhouse conditions

| Transgenic line | Days to flowering in the | Days to flowering in the |
|---|---|---|
| ID | phytotrone | greenhouse |
| Excellent Flair | Did not flower | 139.0 ± 7.4 |
| PHY A | | |
| HA7 | 84.5 ± 0.6 | 108.3 ± 1.3 |
| HA4 | 93.3 ± 3.4 | 112.3 ± 2.1 |
| HA16 | 86.3 ± 6.4 | 109.0 ± 2.5 |
| HA25 | 95.6 ± 9.1 | 106.8 ± 1.2 |
| HA23 | 91.5 ± 2.8 | 111.1 ± 2.0 |
| PHY B | | |
| HB25 | 113.5 ± 3.0 | 108.4 ± 1.6 |
| HB31 | 93.5 ± 5.9 | 111.4 ± 3.6 |
| HB16 | 99.0 ± 1.2 | 108.4 ± 3.1 |
| HB19 | 98.5 ± 7.1 | 107.0 ± 5.4 |
| HB42 | 84.3 ± 3.1 | 110.0 ± 2.3 |

As is evident from this data, the PHY A or PHY B transgenic Hypericum plants developed shoots and reached flowering under the short day conditions provided in the phytotrone. In contrast, these conditions were insufficient for the non-transgenic Hypericum cv. “Excellent Flair” plants, and as such these plants developed shoots but did not flower.

The transgenic and non-transgenic plants reacted differently to the gradual increase in day length provided by the greenhouse conditions. Although all of the rooted cuttings were planted in February, the PHY A or PHY B transgenic plants lines flowered in May whereas the non-transgenic plants flowered approximately one month later.

These results clearly demonstrate that flower initiation in the transgenic Hypericum plant lines generated according to the teachings of the present invention occurs under day length conditions which are substantially shorter than that needed for flowering of similar non-transgenic plants. This is clearly demonstrated under greenhouse conditions in which a gradual increase in day-length over time supported flowering in the transgenic plant lines a full month before enabling flowering of identical but non-transformed plants.

These results are further demonstrated in FIG. 5, which illustrates the growth state of greenhouse grown transgenic and non-transgenic lines in June (day length of approximately 14 hours). At this stage, transgenic plant fruits are clearly evident while the equivalent non-transgenic plants are still at their flowering stage necessitating an additional growth period for fruit development.

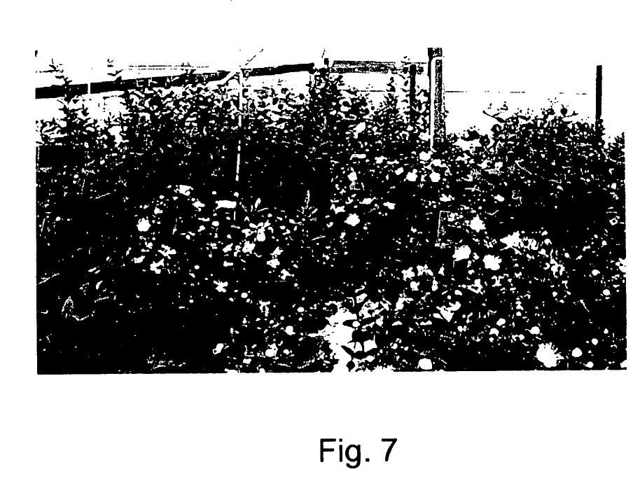

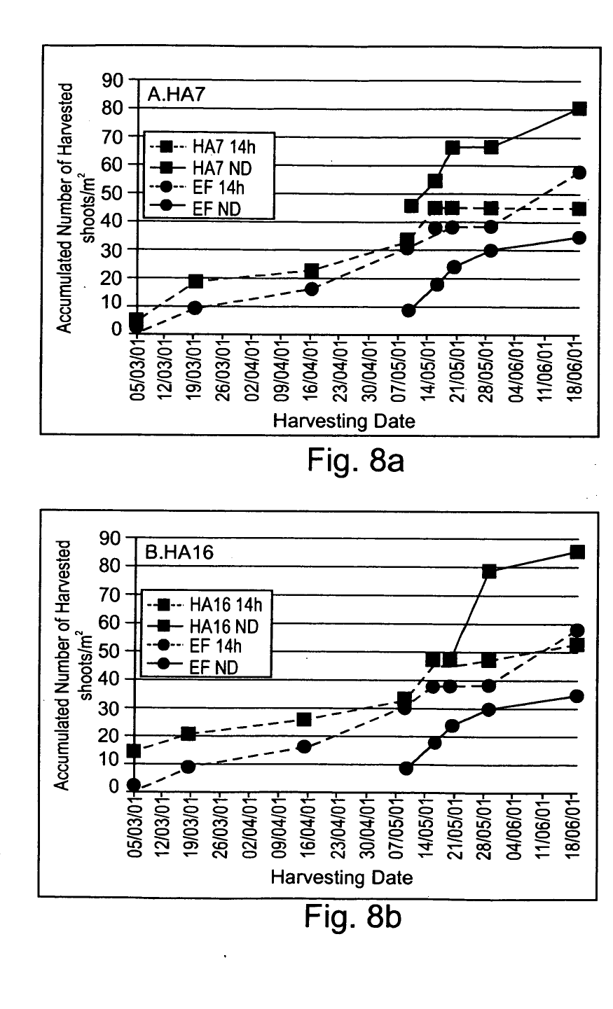

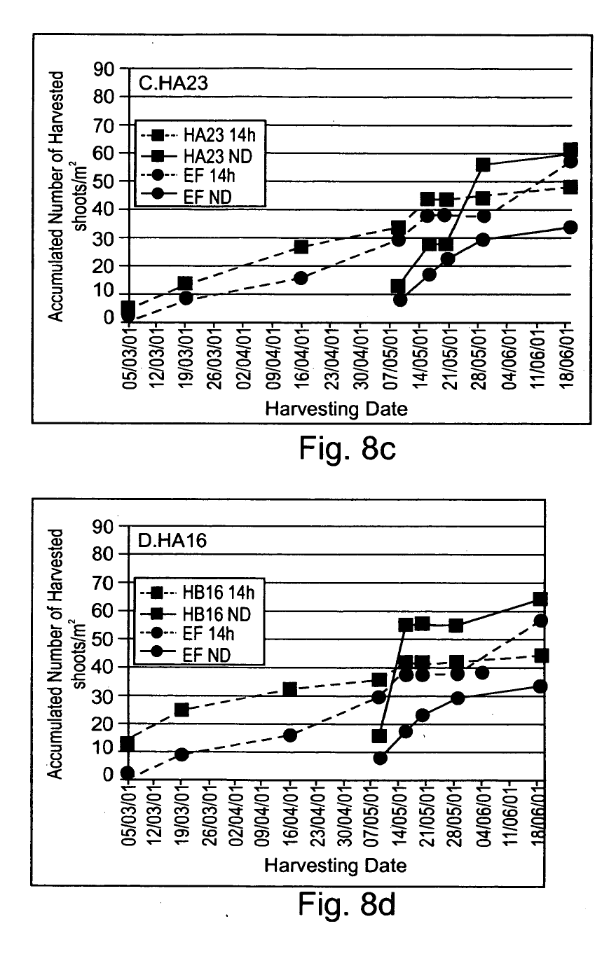

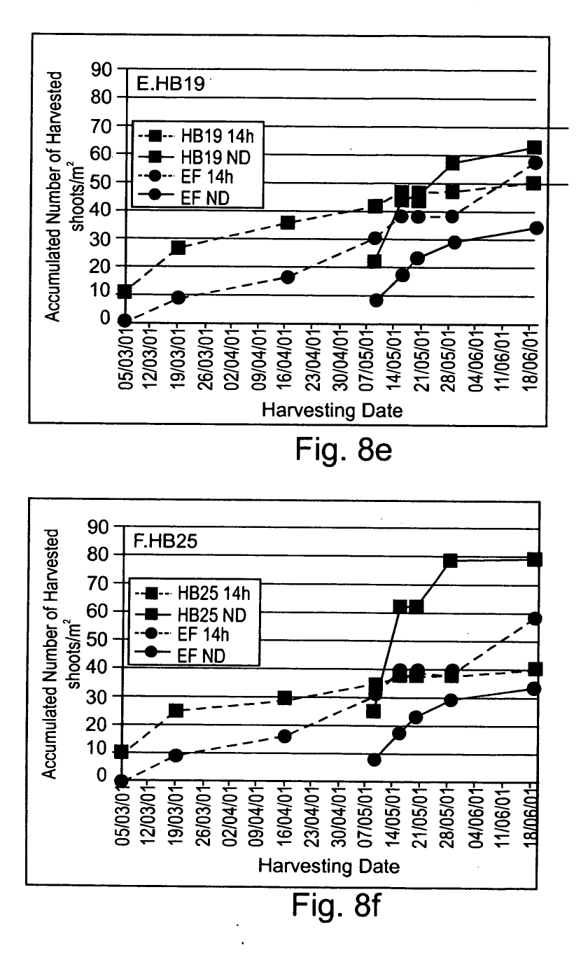

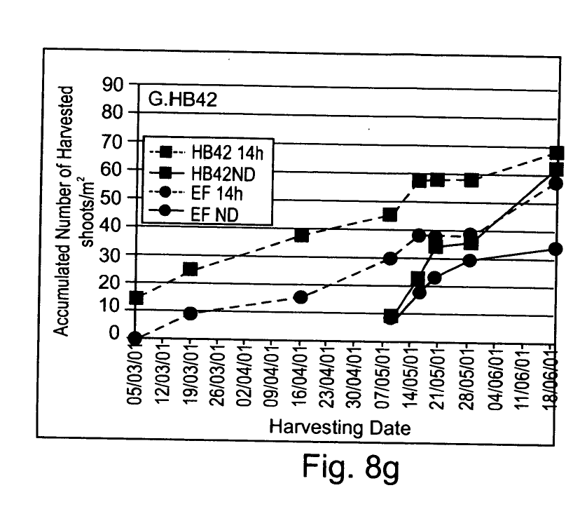

In the field, flowering and fruit setting in transgenic Hypericum plants preceded flowering and fruit setting of the wild type “Excellent Flair” plants (FIG. 7). Therefore phytochrome overexpression in Hypericum advanced the appearance of fruit bearing shoots especially under natural day-length conditions (FIGS. 8a-g) and thus commercial yield of transgenic Hypericum plants preceded that of the wild type plants by nearly one month.

Thus, the present invention provides two genetically distinct plant species which when over expressing phytochrome A or B respond in a qualitative manner to day length manipulation.

Aster (Asteraceae) cv “Sun Karlo” is an herbaceous perennial plant cultivated for its flowering shoot. Wild type Aster plant requires long-day conditions for its inflorescent-shoot development, which on the other hand retards further flower development of the inflorescent shoot. As exemplified hereinabove, phytochrome overexpressing Aster plants responded to day length manipulation in a qualitative manner manifested by induction of inflorescent-shoot development.

Hypericum (guttiferae) cv “Excellent Flair” is a woody perennial plant, cultivated for its decorative fruits. Wild type Hypericum requires long-day conditions for flower initiation. As exemplified hereinabove, phytochrome overexpressing Hypericum plants responded to day length manipulation in a qualitative manner manifested by flowering initiation under substantially short day conditions.

In any case, plants generated according to the teachings of the present invention are particularly suitable for commercial cultivation since they respond, in flowering, to substantially shorter days thus enabling commercial cultivation year round with little or no need for artificial lighting.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### References

### (Additional References are Cited in the Text)

1. Bagnall D. J., King R. W., Whitelam G. C., Boylan M. T., Wagner D. and Quail P. H., 1995. Flowering response to altered expression of phytochrome in mutants and transgenic lines of Arabidopsis thaliana (L.) Heynh. Plant Physiol. 108, 1495-1503.
2. Bernier G.,Kinet J. M. and Sachs R. M., 1981.The Physiology of Flowering. CRC press, Boca Raton Fla.
3. Casal J. J., Clough R. C. and Viestra R. D., 1996. High-irradiance responses induced by far-red light in grass seedlings of the wild type or overexpressing phytochrome A. Planta 200, 132-137.
4. Goto N., Kumagai T. and Koornneef M., 1991. Flowering responses to light-breaks in photomorphogenic mutants of Arabidopsis thaliana, a long-day plant. Physiol. Plant. 83, 209-215.
5. Halevy A. H., 1985. Handbook of Flowering. CRC press Boca Raton, Fla.
6. Johanson E., Bradley M., Harberd N. P. and Whitelam G. C., 1994. Photoresponses of light grown phy-A mutants of Arabidopsis. Plant physiol. 105, 141-149.
7. Mathews S. and Sharrock R. A., 1997. Phytochrome gene diversity. Plant, Cell and Environment 20, 666-672.Murtas G. and Millar A. J., 2000. How plants tell the time. Current Opinion in Plant Biology, 3, 43-46.
8. Putterill J., Robson F., Lee K., Simon R. and Copland G., 1995. The CONSTANT gene of Arabidopsis promotes flowering and encodes a protein showing similarities to zink finger transcription factors. Cell, 80, 847-857.
9. Reed J. W., Nagpal P., Poole D. S., Furuya M. and Chory J., 1993. Mutation in the gene for the Red/Far-red light receptor phytochrome B alter cell elongation and physiological responses throughout Arabidopsis development. The Plant Cell, 5, 147-157.
10. Reed J. W., Nagatani A., Elich T. D., Fagan M. and Chory J., 1994. Phytochrome A and phytochrome B have overlapping but distinct functions in Arabidopsis development. Plant physiol. 104, 1139-1149.
11. Samach A. and Copland G., 2000. Time measurements and the control of flowering in plants. Review articles BioAssay 22, 38-47
12. Somer D. E., Devlin P. F. and Kay S. A., 1998. Phytochrome and cryptochromes in the entrainment of the Arabidopsis circadian clock. Science 282, 1488-1490.
13. Thomas B. and Vince-Prue D., 1995. Do long-day plants and short-day plants perceive day length in the same way? FNL 20, 50-57.
14. Thomas B. and Vince-Prue D., 1997. Photoperiodism in Plants. Academic Press, London.
15. Vince-Prue D, 1994. The duration of light and photoperiodic responses. In: Photomorphogenesis in Plants. Kendrick R. E. and Kronenberg G. H. M., eds. Kluwer, press Amsterdam. Pp. 447-490.
16. Weller J. L., Murfet I. C. and Reid J. B., 1997. Pea mutants with reduced sensitivity to Far-red light define an important role for phytochrome A in day-length detection. Plant physiol. 114, 1225-1236.
17. Whitelam G. C. and Harberd N. P., 1994. Action and function of phytochrome family members revealed through the study of mutant and transgenic plants. Plant Cell and Environment 17, 615-625.
18. Whitelam G. C. and Devlin P. E., 1997. Roles of different phytochrome in Arabidopsis photomorphogenesis. Plant, Cell and Environment 20, 752-759.

1. A long day plant cultivated for commercial production of flowering-shoots, flowering pots, flowers, seeds or fruits, the long day plant overexpressing a phytochrome protein in at least a portion of it's cells, such that said flowering-shoots, flowering pots, flowers, seeds or fruits thereof develop under substantially shorter days than that required for development of said flowering-shoots, flowering pots, flowers, seeds or fruits in a similar long day plant not over expressing said phytochrome protein.

2. The long day plant of claim 1, wherein said substantially shorter days are effected by natural lighting conditions.

3. The long day plant of claim 1, wherein said substantially shorter days are further characterized by at least one condition selected from the group consisting of a light intensity between 80-2000 μmole m⁻²s⁻¹ PAR, and a temperature selected from the range between 5-30° C.

4. The long day plant of claim 1, wherein the long day plant overexpressing said phytochrome protein is derived from a commercial plant, plant derived tissue or a plant cell transformed with an exogenous expression cassette for overexpressing said phytochrome protein.

5. The long day plant of claim 4, wherein said commercial plant, plant derived tissue or a plant cell is stably or transiently transformed with said expression cassette for overexpressing said phytochrome protein.

6. The long day plant of claim 5, wherein said expression cassette forms a part of a nucleic acid construct selected from the group consisting of a DNA construct or an RNA construct.

7. The long day plant of claim 4, wherein said exogenous expression cassette includes a phytochrome A or a phytochrome B encoding sequences.

8. The long day plant of claim 7, wherein said exogenous expression cassette also includes a promoter sequence for directing expression of said phytochrome A or said phytochrome B encoding sequences in plant tissue.

9. The long day plant of claim 8, wherein said promoter is selected from the group consisting of a constitutive promoter, an inducible promoter a developmentally regulated promoter and a tissue specific promoter.

10. The long day plant of claim 1, wherein the long day plant overexpressing said phytochrome protein is a commercial dicotyledonous or monocotyledonous plant.

11. The long day plant of claim 1, wherein the long day plant overexpressing said phytochrome protein is selected from the group consisting of an agronomic crop, a horticultural crop, and an ornamental plant.

12. The long day plant of claim 1, wherein the long day plant overexpressing said phytochrome protein is an annual or a perennial plant selected from the group consisting of a rosette forming plant, a bulb forming plant, a corm forming plant, an herbaceous plant, a shrub forming plant and a tree forming plant.

13. The long day plant of claim 1, wherein said flowering-shoots, flowering pots, flowers, seeds or fruits which develop under said substantially shorter days have at least one improved agronomic and/or commercial characteristic selected from the group consisting of an increased number of flowering shoots, an increased number of flowers, an increased number of fruit forming flowers and a faster growth rate, lower cold request for growth and flowering, and reduced light intensity dependent flowering as compared to said similar long day plant not over expressing said phytochrome protein.

14. The long day plant of claim 1, wherein a day length of said substantially shorter days is at least 15% shorter than that required by said similar long day plant not over expressing said phytochrome protein.

15. A method of modulating a responsiveness of a long day plant to day length comprising:
(a) overexpressing a phytochrome protein in a plurality of long day plants; and
(b) selecting plants from said plurality of long day plants which are capable of developing flowering-shoots, flowering pots, flowers, seeds or fruits when grown under substantially shorter days than those required for development of said flowering-shoots, flowering pots, flowers, seeds or fruits in a similar long day plant not overexpressing said phytochrome protein, thereby modulating a responsiveness of the long day plant to day length.

16. The method of claim 15, wherein said substantially shorter days are effected by natural lighting conditions.

17. The method of claim 15, wherein the long day plant is cultivated for commercial production of flowering-shoots, flowering pots, flowers, seeds or fruits.

18. The method of claim 15, wherein the long day plant is derived from a commercial plant, plant derived tissue or a plant cell transformed with an exogenous expression cassette for overexpressing said phytochrome protein.

19. The method of claim 18, wherein said commercial plant, plant derived tissue or a plant cell is stably or transiently transformed with said exogenous expression cassette for overexpressing said phytochrome protein.

20. The method of claim 18, wherein said exogenous expression cassette forms a part of a nucleic acid construct selected from the group consisting of a DNA construct or an RNA construct.

21. The method of claim 18, wherein said exogenous expression cassette for overexpressing said phytochrome protein includes a phytochrome A or a phytochrome B encoding sequences.

22. The method of claim 21, wherein said exogenous expression cassette also includes a promoter sequence for directing expression of said phytochrome A or said phytochrome B encoding sequences in plant tissue.

23. The method of claim 22, wherein said promoter is selected from the group consisting of a constitutive promoter, an inducible promoter, a developmentally regulated promoter and a tissue specific promoter.

24. The method of claim 15, wherein a day length of said substantially shorter days is at least 15% shorter than that required by said similar long day plant not over expressing said phytochrome protein.

25. The method of claim 18, wherein said exogenous expression cassette for overexpressing said phytochrome protein is compatible for propagation in cells, or integration into the genome, of a plant.

26. The method of claim 19, wherein said stable and/or said transient plant transformation is followed by identifying said long day plants.

27. The method of claim 15, wherein said overexpressing is effected by transforming said long day plant with an expression cassette encoding a phytochrome protein.

28. The method of claim 27, wherein said phytochrome protein is phytochrome A or phytochrome B.

29. The method of claim 15, wherein modulating the responsiveness of the long day plant to day length is utilized for producing flowering-shoots, flowering pots, flowers, seeds or fruits in the long day plant during substantially shorter days than those required by the long day plant for producing said flowering-shoots, flowering pots, flowers, seeds or fruits.

30. The method of claim 15, wherein modulating the responsiveness of the long day plant to day length is utilized for causing a spring or summer flowering plant to flower during autumn, winter or year-round.

31. The method of claim 15, wherein modulating the responsiveness of the long day plant to day length is utilized for conferring early flowering in the long day plant under short-day conditions.

32. The method of claim 15, wherein said substantially shorter days are further characterized by at least one condition selected from the group consisting of a light intensity between 80-2000 μmole m⁻²s⁻¹ PAR, and a temperature selected from a range between 5-30° C.

33. A method of commercially cultivating a long day plant under short day conditions, comprising:
(a) overexpressing a phytohormone protein in at least a portion of the cells of the long day plant; and
(b) cultivating the long day plant resulting from step (a) under substantially shorter days than those required for development of flowering-shoots, flowering pots, flowers, seeds or fruits in a similar long day plant not overexpressing said phytochrome protein, thereby commercially cultivating said long day plant under the short day conditions.

34. The method of claim 33, wherein said substantially shorter days are effected by natural lighting conditions.

35. The method of claim 33, wherein said long day plant is derived from a commercial plant, plant derived tissue or a plant cell transformed with an exogenous expression cassette for overexpressing said phytochrome protein.

36. The method of claim 35, wherein said commercial plant, plant derived tissue or a plant cell is stably or transiently transformed with said exogenous expression cassette for overexpressing said phytochrome protein.

37. The method of claim 35, wherein said exogenous expression cassette forms a part of a nucleic acid construct selected from the group consisting of a DNA construct or an RNA construct.

38. The method of claim 35, wherein said exogenous expression cassette for overexpressing said phytochrome protein includes a phytochrome A or a phvtochrome B encoding sequences.

39. The method of claim 38, wherein said exogenous expression cassette further includes a promoter sequence for directing an expression of said phytochrome A or said phytochrome B encoding sequences in a plant tissue.

40. The method of claim 39, wherein said promoter is selected from the group consisting of a constitutive promoter, an inducible promoter, a developmentally regulated promoter and a tissue specific promoter.

41. The method of claim 33, wherein a day length of said substantially shorter days is at least 15% shorter than that required by said similar long day plant not overexpressing said phytochrome protein.

42. The method of claim 35, wherein said exogenous expression cassette for overexpressing said phytochrome protein is compatible for propagation in cells, or integration into the genome, of a plant.

43. The method of claim 36, wherein said stable and/or said transient plant transformation is followed by identifying said long day plants.

44. The method of claim 33, wherein said overexpressing is effected by transforming said long day plant with an expression cassette encoding said phytochrome protein.

45. The method of claim 44, wherein said phytochrome protein is phytochrome A or phytochrome B.

46. The method of claim 33, wherein said cultivating is effected during autumn, winter or year-round.

47. The method of claim 33, wherein said substantially shorter days are further characterized by at least one condition selected from the group consisting of a light intensity between 80-2000 μmole m⁻²s⁻¹ PAR, and a temperature selected from a range between 5-30° C.

48. The method of claim 33, wherein the short day conditions are natural short day conditions.


SEQUENCE LISTING (4 sequences)

SEQ ID NO: 1 - DNA, 20 bp, Artificial sequence
  Single strand DNA oligonucleotide
cgccttctggctatcagatg

SEQ ID NO: 2 - DNA, 20 bp, Artificial sequence
  Single strand DNA oligonucleotide
cgaggaagcattgctactgt

SEQ ID NO: 3 - DNA, 22 bp, Artificial sequence
  Single strand DNA oligonucleotide
gcttgttccagcaaggactact

SEQ ID NO: 4 - DNA, 19 bp, Artificial sequence
  Single strand DNA oligonucleotide
gctgctatggaacatgtct